(19) United States
(12) Patent Application Publication
(10) Pub. No.: US 2021/0362690 A1
(43) Pub. Date: Nov. 25, 2021

(54) SHOPPING CART WASHING SYSTEM

(71) Applicant: COREY DION MIDDLETON, SPRING, TX (US)

(72) Inventor: COREY DION MIDDLETON, SPRING, TX (US)

(21) Appl. No.: 17/326,073

(22) Filed: May 20, 2021

Related U.S. Application Data
(60) Provisional application No. 63/027,903, filed on May 20, 2020.

(51) Int. Cl. B60S 3/04; B60S 3/00; B08B 3/02; B08B 5/02; B08B 3/10; A61L 2/26; A61L 2/18; A61L 2/22; A61L 2/10

(52) U.S. Cl. CPC B60S 3/04; B60S 3/002; B08B 3/022; B08B 5/023; B62B 3/14; A61L 2/26; A61L 2/18; A61L 2/22; A61L 2/10; B08B 3/10

(57) ABSTRACT

A washing system for use with a shopping cart includes at least one cleaning solution dispensing unit within a housing that is configured to broadcast a solution, spray, fog, or light onto at least one surface of the shopping cart. The system is configured to cleanse the cart and optionally sanitize a surface of the cart from transferable diseases and illnesses. The system may include a pump, a solution of liquid, a light emitting UV light, a heater and so forth. The dispensing unit may be a pressure washer and a sanitizer. Optionally a dryer may be included to dry the shopping carts as they pass through the housing of the washing system.

### CROSS REFERENCE TO RELATED APPLICATIONS:

This application claims the benefit of an earlier filing date and right of priority to U.S. Provisional Application No. 63/027,903, filed 20 May 2020, the contents of which is incorporated by reference herein in its entirety.

### BACKGROUND OF THE INVENTION

### 1. Field of the Invention

The present application relates to an automated cleaning system, and more particularly to a system for the cleaning of shopping carts.

### 2. Description of Related Art

The need of a cart or portable container is a necessity when shopping. The cart is used to carry various items for the shopper as the shopper travels about the store. Carts come in different styles and types. For example, carts can be carried by the shopper much like a basket. Other carts, most frequently, are pushed by the shopper and roll along the ground. An issue that arises is the cleanliness of the shopping carts. These are used and reused by different shoppers each day. The shoppers grab the handles and move the cart as needed. Germs are prevalent and the spread of such germs can become a serious problem.

Some measures have been taken to try and clean shopping carts. One method is to wipe down portions of the cart with a sanitizing wipe. Given the number of touched areas on the cart and the mesh surfaces; this is extremely time consuming and difficult to ensure adequate cleaning. Other methods try to scan or use light to shine on the carts. These are cumbersome and often require a large amount of time to do as each cart is typically done individually. This naturally hinders the ability to handle large scale uses.

Although strides have been made with respect to cleaning of carts to minimize the exchange of germs, shortcomings remain. It is desired that an assembly be provided that is both effective and scalable for large volumes of carts.

### BRIEF SUMMARY OF THE INVENTION

It is an object of the present application to provide a shopping cart washing system configured to clean and/or wash shopping carts in an effort to potentially disinfect contaminants between users. The system is configured to be a stand-alone assembly wherein the shopping carts are inserted into or through at least a portion of the assembly. The assembly broadcasts a spray or fog into the air for communication with surfaces of the shopping cart.

It is a further object of the present application that the system be adjustable to accommodate different configurations and permit a user to manually select which individual systems are operable. A pressure washer may be incorporated to apply a stream of liquid onto the surfaces. A dryer may be included as well as a sanitizer. Any one or more of these may be used independent of another or as a collective combination.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a side view of a cleaning system according to an embodiment of the present application.

FIG. 2 is an exemplary end view of the cleaning system of FIG. 1.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, the shopping cart washing system of the present application is configured to provide a fog or liquid spray within a defined area for communication with one or more surfaces of a shopping cart. The fog or spray may facilitate many uses, such as washing, cleansing, disinfecting, and so forth. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

The cleaning system of the present application is configured to provide a fast and efficient manner of cleaning shopping carts in an effort to minimize the transmission of germs between shoppers. The system is configured to provide at least a partially enclosed area for the passage of a shopping cart. The shopping cart is cleaned through one or more cleansing means prior to exiting. It is desired that the cleaning system of the present application be portable to allow an end user, such as a grocery store or other business, to selectively locate the system for use. Being portable, it can be moved between an operational location and a storage location as needed to permit occasional use. Additionally, it is conceived that the system may be movable between store locations in instances perhaps where a single store chain has multiple stores that need to share it.

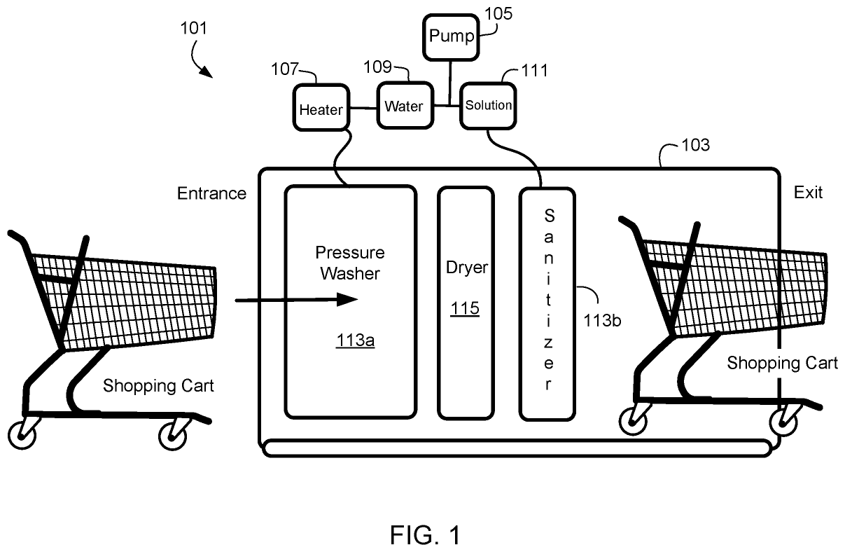

Referring now to FIG. 1 in the drawings, a cleaning system 101 for shopping carts is provided. System 101 is configured to provide a housing 103, and at least one cleaning assembly. Each cleaning assembly may include a liquid solution, a method of dispersing such solution (dispensing unit), and even a pump to pressurize the solution. System 101 is configured to operate with liquid solutions that are sprayed over the surfaces of the shopping cart. In other embodiments, any airborne matter may be dispensed from the cleaning assemblies within the system 101. A fog or liquid mist for example may be used.

Housing 103 is configured to define a confined volume of space in which the solutions of the cleaning assemblies are used. The precise length of housing 103 may be varied depending on many factors, including any of the following: the number of carts to be cleaned simultaneously, the number of cleaning assemblies, and environmental conditions. Housing 103 may have different external and internal sizes to accommodate the shopping carts. The materials used to create housing 103 also may vary and can include any of aluminum, steel, plastic, or composite materials for example.

In operation, a shopping cart is passed through the entrance and is moved through the one or more cleaning assemblies until it passes out the exit. Confinement of the cleaning assemblies within housing 103 acts to avoid disturbances from wind or the elements. Additionally, an advantage of housing 103 is the ability to more effectively control the distribution and coating of the solutions used in the cleaning assemblies. The smaller the volume defined by housing 103, the greater the efficiency of the system.

It should be stated that the use of a solution has many advantages over the act of physically wiping surfaces. There are far too many surfaces to ensure that physical wiping will account for. Use of a sprayed solution allows a universal coating and application to all surfaces of the cart.

A cleaning assembly is a compilation of devices used to induce a cleaning spray or mist or fog that is applied to the shopping cart. As stated, one or more cleaning assemblies may be used. A pump 105 may be used to create a pressure on a liquid solution or water. A heater 107 is optionally included as well to preheat the liquid solution prior to reaching the shopping cart. The liquid solution may be that of water 109, a soap, a chemical cleaner 111, and so forth. Any of the solutions may optionally be used with one or more cleaning assemblies. More than one liquid solution may be used. As seen in FIG. 1, a sanitizer and a pressure washer are used: one using a chemical solution 111 while the other uses a water-based solution 109.

Within housing 103, at least a portion of the cleaning solution dispensing units 113a and 113b are located. It is understood that such units will include one or more nozzles to aerate or form a mist/fog/spray from the pressurized solutions to which it is connected. Each dispensing unit 113a/113b may be used with one or multiple cleaning assemblies, wherein a single nozzle may be used with multiple cleaning liquid solutions for example.

Cleaning solution dispensing unit 113b is configured to decrease the contaminants located on at least one surface of the shopping cart. As noted previously, unit 113b may emit a liquid solution such as a sanitizing spray or mist. Unit 113b may also emit an airborne substance of various types that contacts one or more surfaces of the shopping cart. In some embodiments, unit 113b emits a UV light configured to remove selected contaminants or living microorganisms from a surface.

An optional dryer 115 is used to remove liquid residue from the shopping carts. Dryer 115 and the dispensing units 113a/113b may be located in any particular order and in any location within housing 103. Likewise, it should be understood, housing 103 may be subdivided into separate internal bays or left as a singular volume.

A power supply would be needed to run the various systems or assemblies used in the cleaning system 101. One or more user interfaces will be provided to grant a user operational control over the various assemblies and systems therein. A user may elect to run a single cleaning assembly when multiple are available. Conversely a user may elect to run the dryer or not to run the dryer. A control system will be included to facilitate smooth operational control.

Of note, system 101 in its base form is manually operated wherein a user pushes or pulls the carts(s) through the system 101. One or more may be sent through at a single time. Optionally system 101 may include an automated track assembly configured to transport one or more carts simultaneously through housing 103. This may be a mechanical and/or electronically controlled device. A user merely places the cart in the entrance to engage the track assembly and begin a cleaning cycle. The track assembly carries the cart through the housing.

It is conceived that use of the cleaning system 101 may be primarily used by retailers for high volume cleaning of shopping carts. It is understood that system 101 may be used in different manners. For example, the retailer may elect to choose the frequency of cleaning. Shopping carts may be cleaned when retrieved from lots. Carts may be sent individually through or collectively in a bundle. In another example, system 101 may be used individually by shoppers at the time shopping begins. Shopping carts may be pre-staged from cleaning on demand for shoppers to use. Other ways are conceivable and those mentioned herein are not meant to be limiting.

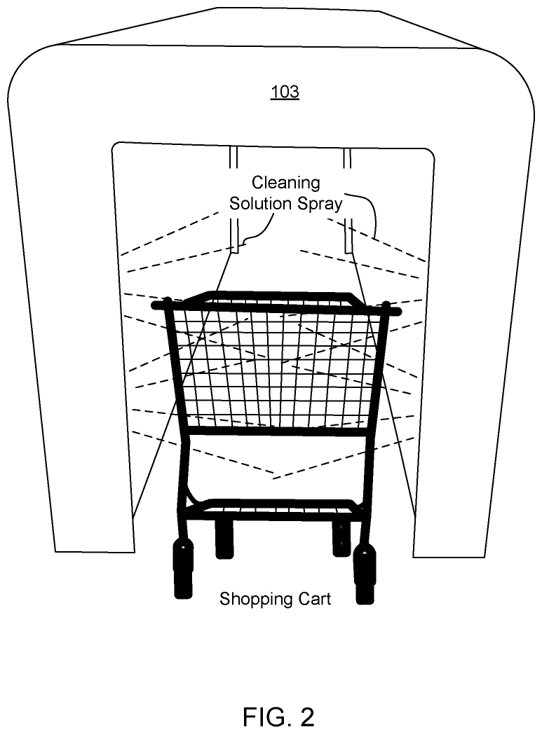

Referring now also to FIG. 2 in the drawings, an end view of an exemplary housing 103 is provided wherein the shopping cart is located at the entrance. The size and shape of the housing 103 is not limiting and is provided for contextual purposes only. A cleaning solution is shown being sprayed internally within housing 103.

The current application has many advantages over the prior art including at least the following: (1) the ability to clean multiple shopping carts simultaneously or in an assembly line fashion to make the act of cleaning them scalable to larger volumes; (2) decreased cleaning times; (3) increased cleaning efficiency; and (4) portability for the retailer/user.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A washing system for use with a shopping cart, comprising:
a housing having an opening;
a washer partially located within the housing; and
a pump in communication with the washer so as to produce a discharge of liquid from the washer;
wherein the liquid contacts the shopping cart as it passes through the housing.

2. The system of claim 1, further comprising:
a heater in communication with the liquid prior to the washer and configured to heat the liquid.

3. The system of claim 1, further comprising:
a dryer configured to apply at least one of a heat and forced air onto the shopping cart after the washer.

4. The system of claim 3, further comprising:
a sanitizer configured to decrease contaminants from at least one surface of the shopping cart.

5. The system of claim 4, wherein the sanitizer emits a solution.

6. The system of claim 4, wherein the sanitizer emits an airborne substance to contact the shopping cart.

7. The system of claim 4, wherein the sanitizer emits a light.

8. The system of claim 1, further comprising:
a sanitizer configured to decrease contaminants from at least one surface of the shopping cart.

9. The system of claim 8, wherein the sanitizer emits a solution.

10. The system of claim 8, wherein the sanitizer emits an airborne substance to contact the shopping cart.

11. The system of claim 8, wherein the sanitizer emits a light.

12. A washing system for use with a shopping cart, comprising:
a housing having an opening;
a sanitizer partially located within the housing and configured to decrease contaminants from at least one surface of the shopping cart; and
a pump in communication with the washer so as to produce a discharge of liquid from the washer;
wherein the liquid contacts the shopping cart as it passes through the housing.

13. The system of claim 12, further comprising:
a dryer configured to apply at least one of a heat and forced air onto the shopping cart after the washer.

14. The system of claim 12, wherein the sanitizer emits a solution.

15. The system of claim 12, wherein the sanitizer emits an airborne substance to contact the shopping cart.

16. The system of claim 12, wherein the sanitizer emits a light.
